(19) United States
(12) Patent Application Publication
(10) Pub. No.: US 2020/0360175 A1
(43) Pub. Date: Nov. 19, 2020

(54) SYSTEMS AND METHODS OF PERIOPERATIVE WARMING

(71) Applicant: Mercury Biomed, LLC, Cleveland, OH (US)

(72) Inventors: James M. Kuras, Macedonia, OH (US); R. Brian Patrick, Houston, TX (US)

(21) Appl. No.: 16/723,453

(22) Filed: Dec. 20, 2019

Related U.S. Application Data
(63) Continuation of application No. 15/703,230, filed on Sep. 13, 2017, now abandoned.
(60) Provisional application No. 62/393,858, filed on Sep. 13, 2016.

(51) Int. Cl. A61F 7/00; A61F 7/02

(52) U.S. Cl. CPC A61F 7/007; A61F 7/02; A61F 2007/0045; A61F 2007/0071; A61F 2007/0093; A61F 2007/0096; A61F 2007/0036; A61F 2007/0288; A61F 2007/0011

(57) ABSTRACT

Described are systems and methods of perioperative warming. One method comprises providing a regulated power supply that is controlled by a controller, wherein the regulated power supply provides power to one or more polymer positive temperature coefficient (PTC) heating elements, each PTC heating element having a saturation voltage and a corresponding saturation temperature; applying a first voltage to at least one of the one or more PTC heating elements, wherein the first voltage is greater than the saturation voltage; monitoring a temperature of the one of the one or more PTC heating elements while the first voltage is being applied; and regulating voltage applied to the one of the one or more PTC heating elements as the monitored temperature of the one of the one or more PTC heating elements approaches the saturation temperature of the one of the one or more PTC heating elements.

### CROSS REFERENCE TO RELATED APPLICATION

This application is a continuation application of U.S. application Ser. No. 15/703,230 filed Sep. 13, 2017, which claims priority to and benefit of U.S. Provisional Patent Application No. 62/393,858 filed Sep. 13, 2016, both of which are fully incorporated by reference and made a part hereof.

### TECHNICAL FIELD

Aspects of the disclosure relate generally to an improvement in technology for warming a person perioperatively using a warming device.

### BACKGROUND

The term “perioperative” is generally defined as “around the time of operation.” The perioperative period is characterized by a sequence including the time preceding an operation when a patient is being prepared for surgery (“the preoperative period”), followed by the time spent in surgery (“the intraoperative period”), and by the time following an operation when the patient is closely monitored for complications while recovering from the effects of anesthesia (“the postoperative period”). Therapeutic warming is employed during at least the intraoperative period in order to prevent or mitigate a constellation of effects that result from hypothermia. In fact, it is increasingly manifest that maintenance of normothermia perioperatively enhances the prospects for a quick, successful recovery from surgery. The effectiveness of therapeutic warming depends upon delivery of enough heat to a patient's body to raise the patient's core body temperature to, or maintain it within, a narrow range, typically near 37° C. This range is called “normothermic” and a body with a core temperature in this range is at “normothermia.” Hypothermia occurs when the core body temperature falls below 36° C.; mild hypothermia occurs when core body temperature is in the range of 34° C. to 36° C. Therefore, “perioperative therapeutic warming” is warming therapy capable of being delivered during one or more of the perioperative periods for the prevention or treatment of hypothermia.

Up to now, perioperative warming has generally been accomplished by circulating a heated fluid (e.g., air, water, etc.) around portions of a patient's body. However, the need to contain the fluid and to prevent heat loss until the area where heat transfer is desired has complicated these systems and limited their design. Further, it may take a substantial amount of time to warm the fluid or cool it, if needed.

Other existing perioperative warming systems are solid state based using resistive wires as a means to generate heat locally e.g., blankets. This technology, although effective, can be unsafe since the technology produces a constant output regardless of the surroundings, environment or application. Additionally, if the resistive wire circuit breaks it could cause an unintended thermal spike resulting in thermal “run away” creating a risk of burns to the patient.

Therefore, systems and methods are desired that overcome challenges in the art, some of which are described above.

### SUMMARY

An alternative to resistive wires and fluid-based perioperative warming system is a polymer Positive Temperature Coefficient (PTC) heating element-based perioperative warming system and method of use. This type of PTC element offers a number of distinct advantages over traditional resistive wire and fluid heating, particularly safety and durability. The safety features of a PTC heating element perioperative warming system include PTC heaters that are self-regulating in that as temperature rises resistance increases reducing power consumption. This characteristic allows the heater to reach the saturation temperature without the requirement of an external control. Further, PTC heaters are self-limiting in that at a specific applied voltage (vDC), the heater cannot exceed the saturation temperature and does not require an overheat protection circuit.

Also, the PTC heater is inherently durable due to its design. There are no moving parts and in its simplest form are a parallel configured array of crystalline ink-based resistors.

In one aspect, a method of perioperative warming of a patient is disclosed. The method comprises placing one or more polymer positive temperature coefficient (PTC) heating elements in close proximity to a patient's skin, each PTC heating element having a saturation voltage and a corresponding saturation temperature; applying a first voltage to at least one of the one or more PTC heating elements, wherein the first voltage is greater than the saturation voltage; monitoring a temperature of the one of the one or more PTC heating elements while the first voltage is being applied; and regulating voltage applied to the one of the one or more PTC heating elements as the monitored temperature of the one of the one or more PTC heating elements approaches the saturation temperature of the one of the one or more PTC heating elements.

Alternatively or optionally, placing the one or more PTC heating elements in close proximity to the patient's skin may comprise placing the one or more PTC heating elements in one or more pads, and affixing the one or more pads to the patient's skin. For example, the one or more pads may be affixed to each of the patient's feet and/or each of the patient's hands and/or to the patient's neck.

Alternatively or optionally, monitoring the temperature of the one of the one or more PTC heating elements while the first voltage is being applied each pad may comprise monitoring the temperature between the PTC heating element and the patient's skin. For example, the temperature may be monitored using a thermistor, a thermocouple, or the like in communication with a controller.

Alternatively or optionally, regulating the voltage applied to the one of the one or more PTC heating elements as the monitored temperature of the one of the one or more PTC heating elements approaches the saturation temperature of the one of the one or more PTC heating elements may comprise reducing the voltage applied to the one of the one or more PTC heating elements. In one aspect, the voltage may be reduced to the saturation voltage, or lower. Generally, the voltage is regulated using a controller.

In another aspect, an alternative method of perioperative warming of a patient is described. This method comprises providing a regulated power supply that is controlled by a controller, wherein the regulated power supply provides power to one or more polymer positive temperature coefficient (PTC) heating elements in close proximity to a patient's skin, each PTC heating element having a saturation voltage and a corresponding saturation temperature; applying, by the regulated power supply, a first voltage to at least one of the one or more PTC heating elements, wherein the first voltage is greater than the saturation voltage; monitoring, by a feedback loop in communication with the controller, a temperature of the one of the one or more PTC heating elements while the first voltage is being applied; and regulating, by the regulated power supply in communication with the controller, voltage applied to the one of the one or more PTC heating elements as the monitored temperature of the one of the one or more PTC heating elements approaches the saturation temperature of the one of the one or more PTC heating elements.

Another aspect described herein comprises a system for perioperative warming of a patient. One embodiment of the system comprises a regulated power supply; a controller, wherein the regulated power supply is in communication with and controlled by the controller; one or more polymer positive temperature coefficient (PTC) heating elements, wherein the one or more PTC heating elements are connected to the power supply, each PTC heating element having a saturation voltage and a corresponding saturation temperature; and one or more temperature measurement devices in communication with the controller through a feedback loop, wherein a first voltage that is greater than the saturation voltage is applied by the regulated power supply to at least one of the one or more PTC heating elements, a temperature of the one of the one or more PTC heating elements is monitored by the one or more temperature measurement devices in communication with the controller through a feedback loop while the first voltage is being applied, and voltage applied to the one of the one or more PTC heating elements is regulated by the regulated power supply in communication with the controller as the monitored temperature of the one of the one or more PTC heating elements approaches the saturation temperature of the one of the one or more PTC heating elements.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 is an overview illustration of an exemplary system 100 for perioperative warming of a patient;

FIG. 2 shows a flowchart of an embodiment of a method of perioperative warming of a patient;

FIG. 3 is a flowchart that illustrates another method of perioperative warming of a patient;

FIG. 4 is a block diagram illustrating an exemplary operating environment for performing the disclosed methods; and

FIG. 5 is a graphic illustration of an example of the operation of the disclosed system

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices. Furthermore, all or portions of aspects of the disclosed can be implemented using cloud-based processing and storage systems and capabilities.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

Polymer PTC heating elements comprise a crystalline conductive ink on a flexible substrate such as Mylar™. The polymer PTC crystalline compositions generate resistive heat up to a specific temperature. Due to their molecular nature, they are not able to surpass this threshold. The total thermal mass of the system is determined by the shape, size, number and distribution of each individual printed heater element. Because each single resistive element is self-limiting, once the equilibrium temperature is reached, heat will only be applied where it's necessary, generally without complicated electronic controllers or feedback loops. PTC technology can self-regulate operational temperature due to resistance change with temperature. The current draw of the heater decreases as the heater warms and will eventually reduce such that the heater will no longer maintain temperature. As the system cools, the current will rise again causing the heater to maintain an operational temperature.

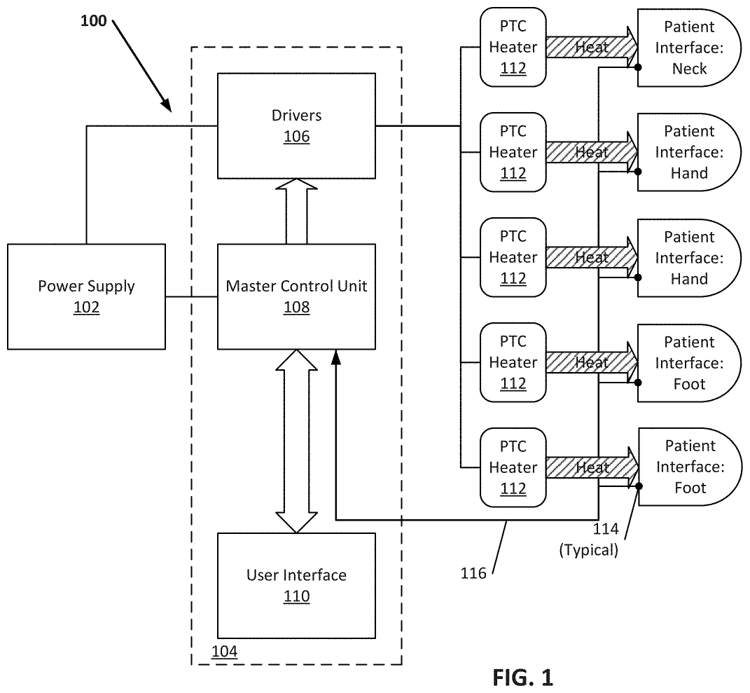

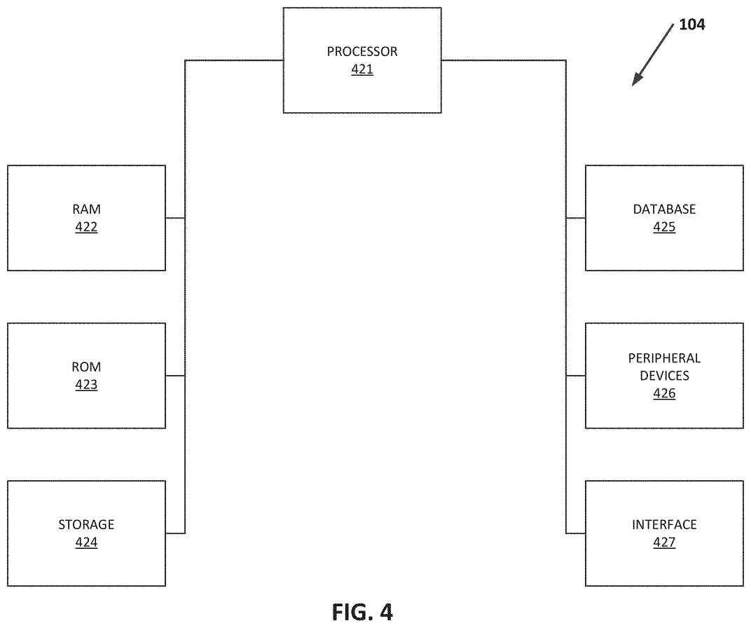

FIG. 1 is an overview illustration of an exemplary system 100 for perioperative warming of a patient. As shown in FIG. 1, the simplified system comprises a regulated power supply 102 and a controller 104, wherein the regulated power supply is in communication with and controlled by the controller. As shown in FIG. 1, the controller 104 comprises drivers 106, master control unit 108 and user interface 110. The controller 104 is described in more detail herein in reference to FIG. 4. The illustrated system 100 further comprises one or more polymer positive temperature coefficient (PTC) heating elements 112. Each of the one or more PTC heating elements 112 are connected to the power supply 102. Each PTC heating element 112 has a saturation voltage and a corresponding saturation temperature, as described herein. The watt density of each PTC heating element 112 can be altered based on patient interface design and subject and environmental thermal loading parameters. One or more of the PTC heating elements 112 are associated with one or more temperature measurement devices 114 in communication with the controller 104 through a feedback loop 116.

In operation of the system of FIG. 1, a first voltage that is greater than the saturation voltage is applied by the regulated power supply 102 to at least one of the one or more PTC heating elements 112. The temperature of the one of the one or more PTC heating elements 112 is monitored by the one or more temperature measurement devices 114 in communication with the controller 104 through the feedback loop 116 while the first voltage is being applied to the PTC heating element 112. Generally, the temperature is monitored between the PTC heating element and the patient's skin using the one or more temperature measurement devices 114. In non-limiting examples, the one or more temperature measurement devices 114 may comprise one or more thermistors or thermocouples in communication with the controller 104 through the feedback loop 116. Exemplary operating temperature ranges of the described system may be from approximately 35.5 C to approximately 43 C.

Voltage applied to the one of the one or more PTC heating elements 112 is regulated by the regulated power supply 102 in communication with the controller 104 as the monitored temperature of the one of the one or more PTC heating elements 112 approaches the saturation temperature of the one of the one or more PTC heating elements 112. In one non-limiting example, voltage applied to the one of the one or more PTC heating elements 112 is reduced when the monitored temperature of the one of the one or more PTC heating elements 112 is at or within two degrees (Celsius) of the saturation temperature of the one of the one or more PTC heating elements 112. Generally, regulating the voltage applied to the one of the one or more PTC heating elements 112 as the monitored temperature of the one of the one or more PTC heating elements 112 approaches the saturation temperature of the one of the one or more PTC heating elements 112 comprises reducing, by the regulated power supply 102 in communication with the controller 104, the voltage applied to the one of the one or more PTC heating elements 112. For example, the applied voltage may be reduced to the saturation voltage of the PTC heating element 112, or lower.

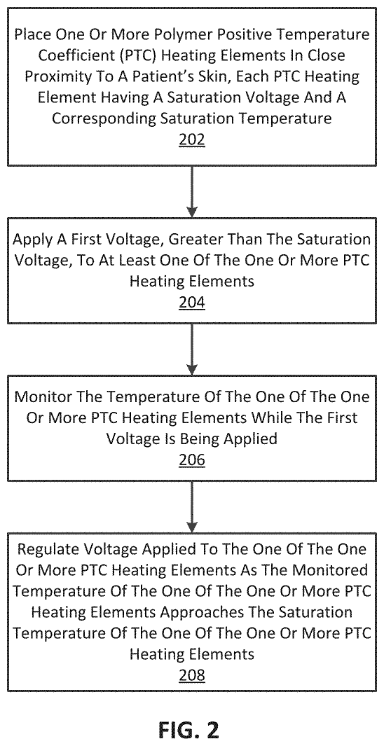

FIG. 2 shows a flowchart of an embodiment of a method of perioperative warming of a patient. The illustrated method comprises 202 placing one or more polymer positive temperature coefficient (PTC) heating elements in close proximity to a patient's skin, each PTC heating element having a saturation voltage and a corresponding saturation temperature. As non-limiting examples, the saturation voltage may be 12 volts DC and the saturation temperature may be 40 Celsius. In one aspect, placing the one or more PTC heating elements in close proximity to the patient's skin may comprise placing the one or more PTC heating elements in one or more pads, and affixing the one or more pads to the patient's skin. In one aspect, the one or more pads may be affixed to each of the patient's feet and/or each of the patient's hands and/or to the patient's neck. In one exemplary embodiment, each pad comprises a plurality of PTC heating elements. These elements may be connected in series or in parallel on a pad. For example, the plurality of heating elements on a pad may be daisy-chained to form a matrix/array that is more flexible than a single PTC heating element such that the pad can conform/deform to the curvature of the applied areas of the body (i.e. hands and feet) resulting in better surface area coverage, intimacy of contact with the skin, and thus superior heat transfer into the body.

At 204, a first voltage is applied to at least one of the one or more PTC heating elements. This first voltage is greater than the saturation voltage such that the temperature of the PTC heating element quickly rises. For example, if the saturation voltage is 12 volts DC, the first voltage may be 18 volts DC. At 206, the temperature of the one of the one or more PTC heating elements is monitored while the first voltage is being applied. Monitoring the temperature of the one of the one or more PTC heating elements while the first voltage is being applied each pad may comprise monitoring the temperature between the PTC heating element and the patient's skin using a feedback loop in communication with a controller. For example, the temperature may be monitored using a thermistor or a thermocouple in communication with a controller. In other aspects, the temperature of the patient, either internally or externally, may be used to control the voltage applied to the PTC heating elements. For example, blood temperature may be monitored and at least partially used to control the voltage applied to one or more of the PTC heating elements.

At 208, voltage applied to the one of the one or more PTC heating elements is regulated as the monitored temperature of the one of the one or more PTC heating elements approaches the saturation temperature of the one of the one or more PTC heating elements. For example, regulating the voltage applied to the one of the one or more PTC heating elements as the monitored temperature of the one of the one or more PTC heating elements approaches the saturation temperature of the one of the one or more PTC heating elements may comprise reducing the voltage applied to the one of the one or more PTC heating elements. Depending upon the approach rate of the temperature to the saturation temperature, the voltage applied to the one of the one or more PTC heating elements may be reduced to the saturation voltage, or lower. Conversely, the voltage applied to the one of the one or more PTC heating elements may be regulated by increasing the voltage, thus driving the temperature toward the saturation temperature even more quickly. The applied voltage is regulated by a controller based on the temperature as monitored by temperature measurement devices that are in communication with the controller through a feedback loop.

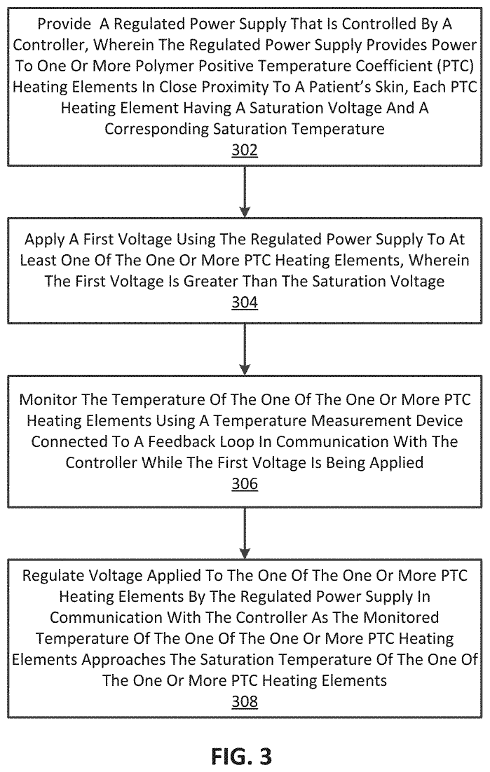

FIG. 3 is a flowchart that illustrates another method of perioperative warming of a patient. The illustrated method comprises 302, providing a regulated power supply that is controlled by a controller, wherein the regulated power supply provides power to one or more polymer positive temperature coefficient (PTC) heating elements in close proximity to a patient's skin, each PTC heating element having a saturation voltage and a corresponding saturation temperature. At 304, a first voltage is applied by the regulated power supply to at least one of the one or more PTC heating elements, wherein the first voltage is greater than the saturation voltage. At 306, a temperature of the one of the one or more PTC heating elements is monitored by temperature measurement devices connected to a feedback loop in communication with the controller, while the first voltage is being applied. At 308, voltage applied to the one of the one or more PTC heating elements is regulated by the regulated power supply in communication with the controller as the monitored temperature of the one of the one or more PTC heating elements approaches the saturation temperature of the one of the one or more PTC heating elements.

The system has been described above as comprised of units. One skilled in the art will appreciate that this is a functional description and that the respective functions can be performed by software, hardware, or a combination of software and hardware. A unit can be software, hardware, or a combination of software and hardware. The units can comprise software in combination with hardware to perform a method for perioperative warming of a patient, as illustrated in FIG. 4 and described below. In one exemplary aspect, the units can comprise a controller 104 as illustrated in FIG. 4, referenced above and described below.

FIG. 4 is a block diagram illustrating an exemplary operating environment for performing the disclosed methods. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The present methods and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise network PCs, minicomputers, mainframe computers, controllers, smartphones, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

FIG. 4 illustrates an exemplary controller 104 that can be used for controlling aspects of a system for perioperative warming of a patient. As used herein, “controller” may include a plurality of controllers. The controllers may include one or more hardware components such as, for example, a processor 421, a random access memory (RAM) module 422, a read-only memory (ROM) module 423, a storage 424, a database 425, one or more peripheral devices 426, and an interface 427. Alternatively and/or additionally, controller 104 may include one or more software components such as, for example, a computer-readable medium including computer executable instructions for performing a method associated with the exemplary embodiments. It is contemplated that one or more of the hardware components listed above may be implemented using software. For example, storage 424 may include a software partition associated with one or more other hardware components. It is understood that the components listed above are exemplary only and not intended to be limiting.

Processor 421 may include one or more processors, each configured to execute instructions and process data to perform one or more functions associated with for controlling aspects of a system for perioperative warming of a patient. Processor 421 may be communicatively coupled to RAM 422, ROM 423, storage 424, database 425, peripheral devices 426, and interface 427. Processor 421 may be configured to execute sequences of computer program instructions to perform various processes. The computer program instructions may be loaded into RAM 422 for execution by processor 421.

RAM 422 and ROM 423 may each include one or more devices for storing information associated with operation of processor 421. For example, ROM 423 may include a memory device configured to access and store information associated with controller 104, including information for identifying, initializing, and monitoring the operation of one or more components and subsystems. RAM 422 may include a memory device for storing data associated with one or more operations of processor 421. For example, ROM 423 may load instructions into RAM 422 for execution by processor 421.

Storage 424 may include any type of mass storage device configured to store information that processor 421 may need to perform processes consistent with the disclosed embodiments. For example, storage 424 may include one or more magnetic and/or optical disk devices, such as hard drives, CD-ROMs, DVD-ROMs, or any other type of mass media device.

Database 425 may include one or more software and/or hardware components that cooperate to store, organize, sort, filter, and/or arrange data used by controller 104 and/or processor 421. For example, database 425 may store historical data related to the temperature of the PTC heating element approaching the saturation temperature, the rate of approach, the differential for reducing and/or regulating voltage applied to the PTC heating element, and the like. Additionally and/or optionally, database 425 may store instructions and/or information to perform a method for perioperative warming of a patient, comprising: placing one or more polymer positive temperature coefficient (PTC) heating elements in close proximity to a patient's skin, each PTC heating element having a saturation voltage and a corresponding saturation temperature; applying a first voltage to at least one of the one or more PTC heating elements, wherein the first voltage is greater than the saturation voltage; monitoring a temperature of the one of the one or more PTC heating elements while the first voltage is being applied; and regulating voltage applied to the one of the one or more PTC heating elements as the monitored temperature of the one of the one or more PTC heating elements approaches the saturation temperature of the one of the one or more PTC heating elements. It is contemplated that database 425 may store additional and/or different information than that listed above.

Peripheral devices 426 may include one or more components configured to communicate information with a user associated with controller 104. For example, peripheral devices 426 may include a console with an integrated keyboard and mouse to allow a user to enter information about a patient and/or to make temperature settings for the perioperative warming of a patient. Peripheral devices 426 may also include a display including a graphical user interface (GUI) for outputting information on a monitor. Peripheral devices 426 may also include devices such as, for example, a printer for printing information associated with controller 104, a user-accessible disk drive (e.g., a USB port, a floppy, CD-ROM, or DVD-ROM drive, etc.) to allow a user to input data stored on a portable media device, a microphone, a speaker system, an image capture device (e.g. camera), or any other suitable type of interface device.

Interface 427 may include one or more components configured to transmit and receive data via a communication network, such as the Internet, Ethernet, a local area network, a wide-area network, a workstation peer-to-peer network, a direct link network, a wireless network, or any other suitable communication platform. For example, interface 427 may include one or more modulators, demodulators, multiplexers, demultiplexers, network communication devices, wireless devices, antennas, modems, and any other type of device configured to enable data communication via a communication network.

### EXAMPLES/RESULTS

In one non-limiting example, a PTC heating element was set (designed) to saturate at 40 C when applied with 12 volt DC (saturation voltage). To ensure the PTC heaters reached the user applied set temperature, the voltage was amplitude modulated affecting both the heating rate and temperature plateau (saturation temperature). This was achieved through the application of a temperature feedback loop (using, for example thermistor or thermocouples to measure temperature) and a proportional—integral—derivative (PID) loop controller.

While the PTC heater element had a designed saturate temperature of 40 C when 12 volts DC was applied, a target temperature (user set) of the pad-patient interface was set to 42.5 C. The controller modulated the voltage applied to the PTC heating element to rapidly reach the set temperature by increasing the applied voltage above the saturation voltage. Once the set temperature was obtained, the controller maintained the set temperature unless the temperature trips a high level set point. When that occurs the voltage will reset to the saturation voltage (e.g., 12 volts DC), causing the PTC heater to regulate to its saturation temperature (e.g., 40 C). Once reached, the PID loop controller step controls the voltage in order to maintain the user setting.

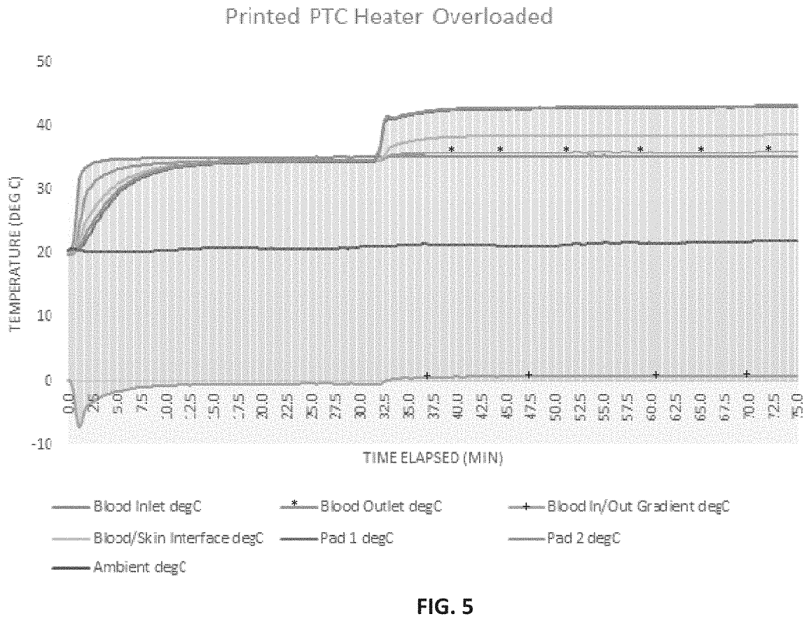

FIG. 5 is a graphic illustration of an example of the operation of the disclosed system. The graph of FIG. 5 illustrates a PTC heater element attached to an apparatus that simulates the pad-patient interface. Referring to the graph, the initial 30 minutes establishes the system steady state temperature of the patient side of the apparatus at 35.5 C (reflecting a hyperthermic patient). At the 30 minute mark the PTC heater element has a voltage applied that is higher than the saturation voltage and once the temperature approaches the saturation temp 40 C the voltage is reduced to 12 volts DC (saturation temperature). The graph illustrates an increase in the steady state temperature after reaching the saturation temperature through an applied voltage (18 volts DC) increase that is above the saturation voltage (12 volts DC). This temperature is sustained through the controller software and feedback loop.

As can be seen in FIG. 5, the blood gradient (marked with a plus sign “+”) is an indication of heat absorption into the “blood side” of the apparatus. The red line on the graph (marked with an asterisk “*”) is the blood flow out and it has increased by ˜1 C to an output of 36.5 C, which is normothermic.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications are referenced. The disclosures of these publications in their entireties are hereby incorporated by reference into this application in order to more fully describe the state of the art to which the methods and systems pertain.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method of perioperative warming of a patient, comprising:
placing one or more polymer positive temperature coefficient (PTC) heating elements in close proximity to a patient's skin, each PTC heating element having a saturation voltage and a corresponding saturation temperature;
applying a first voltage to at least one of the one or more PTC heating elements, wherein the first voltage is greater than the saturation voltage;
monitoring a temperature of the one of the one or more PTC heating elements while the first voltage is being applied; and
regulating voltage applied to the one of the one or more PTC heating elements as the monitored temperature of the one of the one or more PTC heating elements approaches the saturation temperature of the one of the one or more PTC heating elements.

2. The method of claim 1, wherein placing the one or more PTC heating elements in close proximity to the patient's skin comprises placing the one or more PTC heating elements in one or more pads, and affixing the one or more pads to the patient's skin.

3. The method of claim 2, wherein the one or more pads are affixed to each of the patient's feet and/or each of the patient's hands and/or to the patient's neck.

4. The method of claim 1, wherein the saturation voltage is 12 volts DC and the saturation temperature is 40 Celsius.

5. The method of claim 1, wherein the first voltage is 18 volts DC.

6. The method of claim 1, wherein monitoring the temperature of the one of the one or more PTC heating elements while the first voltage is being applied each pad comprises monitoring the temperature between the PTC heating element and the patient's skin.

7. The method of claim 6, wherein the temperature is monitored using a thermistor or a thermocouple in communication with a controller.

8. The method of claim 1, wherein regulating the voltage applied to the one of the one or more PTC heating elements as the monitored temperature of the one of the one or more PTC heating elements approaches the saturation temperature of the one of the one or more PTC heating elements comprises reducing the voltage applied to the one of the one or more PTC heating elements.

9. The method of claim 8, wherein the voltage is reduced to the saturation voltage, or lower.

10. The method of claim 8, wherein the voltage is regulated using a controller.

11. A method of perioperative warming of a patient, comprising:
providing a regulated power supply that is controlled by a controller, wherein the regulated power supply provides power to one or more polymer positive temperature coefficient (PTC) heating elements in close proximity to a patient's skin, each PTC heating element having a saturation voltage and a corresponding saturation temperature;
applying, by the regulated power supply, a first voltage to at least one of the one or more PTC heating elements, wherein the first voltage is greater than the saturation voltage;
monitoring, by a feedback loop in communication with the controller, a temperature of the one of the one or more PTC heating elements while the first voltage is being applied; and
regulating, by the regulated power supply in communication with the controller, voltage applied to the one of the one or more PTC heating elements as the monitored temperature of the one of the one or more PTC heating elements approaches the saturation temperature of the one of the one or more PTC heating elements.

12. The method of claim 11, wherein placing the one or more PTC heating elements in close proximity to the patient's skin comprises placing the one or more PTC heating elements in one or more pads, and affixing the one or more pads to the patient's skin.

13. The method of claim 12, wherein the one or more pads are affixed to each of the patient's feet and/or each of the patient's hands, and/or to the patient's neck.

14. The method of claim 11, wherein the saturation voltage is 12 volts DC and the saturation temperature is 40 Celsius.

15. The method of claim 11, wherein the first voltage is 18 volts DC.

16. The method of claim 11, wherein monitoring the temperature of the one of the one or more PTC heating elements while the first voltage is being applied each pad comprises monitoring the temperature between the PTC heating element and the patient's skin.

17. The method of claim 16, wherein the temperature is monitored using a thermistor or a thermocouple in communication with the controller.

18. The method of claim 11, wherein regulating the voltage applied to the one of the one or more PTC heating elements as the monitored temperature of the one of the one or more PTC heating elements approaches the saturation temperature of the one of the one or more PTC heating elements comprises reducing, by the regulated power supply in communication with the controller, the voltage applied to the one of the one or more PTC heating elements.

19. The method of claim 18, wherein the voltage is reduced to the saturation voltage, or lower.

20. A system for perioperative warming of a patient, comprising:
a regulated power supply;
a controller, wherein the regulated power supply is in communication with and controlled by the controller;
one or more polymer positive temperature coefficient (PTC) heating elements, wherein the one or more PTC heating elements are connected to the power supply, each PTC heating element having a saturation voltage and a corresponding saturation temperature; and
one or more temperature measurement devices in communication with the controller through a feedback loop, wherein a first voltage that is greater than the saturation voltage is applied by the regulated power supply to at least one of the one or more PTC heating elements, a temperature of the one of the one or more PTC heating elements is monitored by the one or more temperature measurement devices in communication with the controller through a feedback loop while the first voltage is being applied, and voltage applied to the one of the one or more PTC heating elements is regulated by the regulated power supply in communication with the controller as the monitored temperature of the one of the one or more PTC heating elements approaches the saturation temperature of the one of the one or more PTC heating elements.

21. The system of claim 20, wherein the one or more PTC heating elements are placed in close proximity to the patient's skin.

22. The system of claim 21, wherein placing the one or more PTC heating in close proximity to the patient's skin comprises placing the one or more PTC heating elements in one or more pads, and affixing the one or more pads to the patient's skin.

23. The system of claim 22, wherein the one or more pads are affixed to each of the patient's feet and/or each of the patient's hands and/or to the patient's neck.

24. The system of claim 20, wherein the saturation voltage is 12 volts DC and the saturation temperature is 40 Celsius.

25. The system of claim 20, wherein the first voltage is 18 volts DC.

26. The system of claim 20, wherein monitoring the temperature of the one of the one or more PTC heating elements while the first voltage is being applied each pad comprises monitoring the temperature between the PTC heating element and the patient's skin using the one or more temperature measurement devices.

27. The system of claim 26, wherein the one or more temperature measurement devices comprise one or more thermistors or thermocouples in communication with the controller through the feedback loop.

28. The system of claim 20, wherein regulating the voltage applied to the one of the one or more PTC heating elements as the monitored temperature of the one of the one or more PTC heating elements approaches the saturation temperature of the one of the one or more PTC heating elements comprises reducing, by the regulated power supply in communication with the controller, the voltage applied to the one of the one or more PTC heating elements.

29. The system of claim 28, wherein the voltage is reduced to the saturation voltage, or lower.
